(19) United States
(12) Patent Application Publication
(10) Pub. No.: US 2021/0156739 A1
(43) Pub. Date: May 27, 2021

(54) Wearable UV Exposure Sensor

(71) Applicant: HALLSTAR BEAUTY AND PERSONAL CARE INNOVATIONS COMPANY, Chicago, IL (US)

(72) Inventors: Shengkui Hu, Darien, IL (US); Hui Feng, Suzhou (CN)

(21) Appl. No.: 16/692,601

(22) Filed: Nov. 22, 2019

(51) Int. Cl. G01J 1/50; A61B 5/00

(52) U.S. Cl. CPC G01J 1/50; A61B 5/6804; A61B 5/6833; A61B 5/681; A61B 5/6803

(57) ABSTRACT

The present disclosure relates, according to some embodiments, to a wearable UV sensor for detecting a dose of an electromagnetic radiation, the wearable UV sensor comprising: (a) a polymer substrate comprising at least one polymer and at least one UV radiation exposure indicator; and (b) an adhesive, wherein exposure of the wearable UV sensor to the dose of the electromagnetic radiation produces a photochromic change in the polymer substrate. The present disclosure also relates to a method for determining a magnitude of a dose of UV radiation exposure, the method comprising: providing a wearable UV sensor comprising a polymer substrate, a UV radiation exposure indicator, and optionally an adhesive layer; exposing the wearable UV sensor to at least one dose of an electromagnetic radiation to form an irradiated wearable UV sensor, wherein the exposing results in a photochromic change by the wearable UV sensor; and determining the magnitude of the at least one dose of electromagnetic radiation.

### FIELD OF THE DISCLOSURE

The present disclosure relates, in some embodiments, to wearable articles sensitive to ultra violet radiation including, for example, a wearable UV exposure indicator for indicating exposure of a body surface to ultra violet radiation.

### BACKGROUND OF THE DISCLOSURE

Increased public awareness regarding the cosmetic and medical hazards associated with exposure of human skin to ultraviolet (UV) radiation has contributed to the increasing popularity of UV absorbing or scattering compounds (sunscreens), both independently and as a component of cosmetic products. However, the effectiveness of sunscreen products to prevent photo damage of skin from the adsorption of UV-B (280-290 nm) and UV-A (320-400 nm) may be hindered by sunscreen layer erosion, faulty sun protection factor (SPF) label characteristics, or wearer compliance.

### SUMMARY

Accordingly, a need has arisen for improved wearable articles sensitive to ultra violet radiation and methods for determining the effectiveness of applied sun protection products. A wearable UV sensor for detecting UV radiation (e.g., a threshold dose of a UV radiation), the wearable UV sensor comprising: a first layer comprising a polymer substrate comprising a UV radiation exposure indicator; and a second layer comprising an adhesive; wherein exposure of the UV radiation exposure indicator to the UV radiation (e.g., the threshold dose of the UV radiation) produces a photochromic response in the UV radiation exposure indicator, wherein the wearable UV sensor is sized to be worn on a human subject, wherein the UV radiation comprises a wavelength range from about 290 nm to about 400 nm, and wherein the wearable UV sensor is configured to receive an applied product in a manner comparable to human skin. Comparable to human skin may comprise a UV sensor being coated by an applied product in the same way as the human skin. Comparable to human skin may comprise a UV sensor receiving an applied product in the same way as the human skin. Comparable to human skin may comprise a UV sensor retaining an applied product in the same way as the human skin.

A wearable UV sensor for detecting UV radiation (e.g., a threshold dose of a UV radiation), the wearable UV sensor comprising a first layer comprising a polymer substrate; and a second layer comprising a UV radiation exposure indicator; wherein exposure of the UV radiation exposure indicator to the UV radiation (e.g., the threshold dose of the UV radiation) produces a photochromic response in the UV radiation exposure indicator, wherein the wearable UV sensor is sized to be worn on a human subject, and wherein the UV radiation comprises a wavelength range from about 290 nm to about 400 nm. A wearable UV sensor may further comprise a third layer, the third layer comprising an amount of an adhesive sufficient to adhere at least a portion of the wearable UV sensor to human skin.

A dermal sensor for detecting a dose of a UV radiation, the dermal sensor comprising: (a) a polymer substrate comprising at least one polymer and at least one UV radiation exposure indicator; and (b) an adhesive, wherein exposure of the dermal sensor to the dose of the UV radiation produces a photochromic response in the polymer substrate, wherein the dermal sensor is sized to be worn on a human subject, and wherein the UV radiation comprises a wavelength range from about 290 nm to about 400 nm.

A method for sensing a dose of a UV radiation exposure, the method comprising: (a) providing a wearable UV sensor comprising a polymer substrate and a UV radiation exposure indicator; (b) exposing the wearable UV sensor to at least one dose of the UV radiation to form an irradiated wearable UV sensor; and (c) measuring a photochromic response of the irradiated wearable UV sensor to the at least one dose of the UV radiation to sense the dose of the UV radiation exposure, wherein the photochromic response results from exposure of UV radiation comprising a wavelength from about 290 nm to about 400 nm. Sensing the dose of the UV radiation may comprise comparing the photochromic response to a chart comprising a UV exposure dose photochromic response. A wearable UV sensor may comprise an adhesive. A method may comprise applying the wearable UV sensor to a skin with the adhesive layer contacting the skin.

A method for determining a magnitude of a UV radiation protection of an applied product, the method comprising: (a) providing a wearable UV sensor comprising a polymer substrate, a UV radiation exposure indicator, and optionally an adhesive layer; (b) contacting a first amount of the applied product to a surface of the wearable UV sensor to produce a contacted sensor surface; (c) exposing the contacted sensor surface to a first dose of a UV radiation to form an irradiated sensor surface, wherein the exposing results in a photochromic change in the irradiated sensor surface; and (d) measuring the photochromic change relative to a reference to determine the magnitude of the UV radiation protection of the applied product by, wherein the first dose of UV radiation comprises a wavelength range from about 290 nm to about 400 nm. A reference may comprise a photochromic change of a surface of the same or another wearable UV sensor (i) not contacted with the applied product and (ii) exposed to the first dose of a UV radiation or a matching dose of UV radiation. A reference may comprise the photochromic change of a surface of the same or another wearable UV sensor (i) contacted with a second amount of the applied product, the second amount being more or less than the first amount, and (ii) exposed to the first dose of a UV radiation or a matching dose of UV radiation. A reference may comprise a chart of photochromic changes of a surface of the same or another wearable UV sensor exposed to a first dose of a UV radiation or a matching dose of UV radiation. Where a wearable UV sensor comprises the optional adhesive layer, a method may comprise applying the wearable UV sensor to a skin with the adhesive layer contacting the skin.

A system for sensing at least one dose of a UV radiation, the system comprising: (a) at least one dermal sensor comprising a polymer substrate, at least one UV radiation exposure indicator, and an adhesive and (b) a reference (e.g., a chart comprising a UV radiation dose response), wherein the at least one dermal sensor is configured to undergo a photochromic response when sensing the at least one dose of the UV radiation, wherein the UV radiation comprises a wavelength from about 290 nm to about 400 nm, and wherein the at least one dermal sensor is sized to be worn on a human subject. A chart may comprise a color gradient, wherein the color gradient corresponds to a UV exposure dose photochromic response. For example, a chart may comprise a color gradient that transitions from a first color to a second color, wherein each step of the color gradient is labeled with a relative UV exposure level. In some embodiments, a UV radiation exposure indicator having a color similar to a first color of a color gradient may have received little to no UV radiation exposure, wherein a corresponding UV radiation exposure indicator having a color similar to a second color of the color gradient may have received a high UV radiation exposure relative to the UV radiation exposure indicator having the color similar to the first color of the color gradient.

A polymer substrate comprises polyethylene, high density polyethylene, low density polyethylene, polypropylene, polyvinyl chloride, poly(methyl methacrylate), nylon, polytetrafluoroethene, polyurea, polystyrene, polysiloxane, polyacrylonitrile, copolymers thereof, or combinations thereof. A UV radiation exposure indicator comprises a photochromic dye comprising spiro-oxazines, spiro-indoline-oazines, spiropyrans, diarylethylenes, azobenzenes, photochromic quinones, inorganic photochromics, photochromic coordination compounds, or combinations thereof. An adhesive may comprise a solvent based adhesive, a polymer dispersion adhesive, a hot-melt adhesive, a contact adhesive, a reactive adhesive, a structural adhesive, an epoxy adhesive, a hot melt adhesive, an elastomeric adhesive, an elastomeric contact adhesive, a melamine formaldehyde adhesive, a phenol formaldehyde adhesive, a polyester resin adhesive, a one-part adhesive, a UV curing adhesive, a moisture curing adhesive, a heat curing adhesive, a vegetable starch adhesive, a casein based adhesive, a cyanoacrylate adhesive, a spray adhesive, a silicone based adhesive, or a combination thereof. A polymer substrate may be present at a concentration from about 70% by weight to about 90% by weight of the wearable UV sensor. A UV radiation exposure indicator may be present at a concentration from about 10% by weight to about 30% by weight of the wearable UV sensor. An adhesive may present at a concentration from about 1% by weight to about 10% by weight of the wearable UV sensor. A wearable UV sensor may have a shape of a ring, a bracelet, a wrist band, an arm band, a necklace, an earring, a belt, an article of clothing, a hat, sunglasses, or a combination thereof. A wearable UV sensor further comprises an area from about 0.1 cm² to about 5 cm². At least one dermal sensor may comprise a pericutaneous sensor having a shape of a ring, a bracelet, a wrist band, an arm band, a necklace, an earring, a belt, an article of clothing, a hat, sunglasses, or a combination thereof. At least one dermal sensor further comprises an area from about 0.1 cm² to about 5 cm². A dose of a UV radiation may be a threshold dose.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

Some embodiments of the disclosure may be understood by referring, in part, to the present disclosure and the accompanying drawings, wherein:

FIG. 1 illustrates an intramolecular rearrangement of a photochromic dye in the presence or absence of ultraviolet radiation and absorbance spectrum data for each form according to a specific example embodiment of the disclosure;

FIG. 2 illustrates photochromic dyes that undergo intramolecular rearrangements in the present of light in the visible or ultraviolet spectrum according to a specific example embodiment of the disclosure;

FIG. 3 illustrates a wearable UV sensor material in the presence and in the absence of ultraviolet radiation according to a specific example embodiment of the disclosure;

FIG. 4 illustrates a wearable UV sensor material in the absence of ultraviolet radiation according to a specific example embodiment of the disclosure;

FIG. 5 illustrates a wearable UV sensor material in the presence of ultraviolet radiation according to a specific example embodiments of the disclosure; and

FIG. 6 illustrates a wearable UV sensor material contacting a skin according to a specific example embodiments of the disclosure.

### DETAILED DESCRIPTION

People have a variety of options from which to choose for protecting themselves from undesirable exposure to harmful radiation while outdoors. These options include a host of products applied or contacted with a subject's skin including lotions, creams, sprays, ointments, and gels, and also including fabrics, garments, shades, cloths, and canopies. Applied products may restrict the capacity for harmful radiation to affect a subject's skin, which may be achieved, for example, by filtering, blocking or otherwise reducing the amount of harmful radiation that reaches the subject. One challenge is knowing the appropriate amount of product to apply to achieve a desired level of protection. Another is assessing how effective a product (e.g., an applied product) is once its in use.

The present disclosure relates, in some embodiments, to wearable articles (including pericutaneous sensors and dermal sensors) sensitive to UV radiation exposure. In some embodiments, wearable UV sensors may be sensitive to or measure an exposure to radiation in the ultraviolet (UV) spectrum comprising UV-A (320-400 nm) radiation and UV-B (280-290 nm) radiation. Wearable UV sensors sensitive to UV radiation may desirably serve as a means for assessing or comparing UV exposure of skin in the absence or presence of an applied product (e.g., sun blocks, UVA filters, or UVB filters). For example, a wearable UV sensor in contact with an applied product may indicate, qualitatively or quantitatively, the extent to which the applied product mitigates UV exposure. Furthermore, a wearable UV sensor for measuring an exposure to UV radiation may provide for a temporal awareness of a dose of UV radiation exposure via observable indicator (i.e., color change). A UV sensor may desirably indicate or display an effectiveness of an applied product in real time. In some embodiments, a UV sensor may be customized to desirably reflect individualized skin along with various application scenarios.

### Wearable UV Sensors

A wearable UV sensor (e.g., a pericutaneous sensor, a dermal sensor) may comprise a substrate (e.g., a polymer substrate); a UV radiation exposure indicator (e.g., photochromic dye); and optionally an adhesive. According to some embodiments, a pericutaneous sensor may be adapted for use near or around the skin of a subject. A wearable UV sensor, in some embodiments, may be adapted for use in contact with (including adhered to) the skin or a subject. Exposure of a wearable UV sensor to UV radiation may induce a photochromic response in the wearable UV sensor, wherein the magnitude of the photochromic response may correlate (e.g., directly correlate) to the magnitude or dose of the UV radiation exposure. A photochromic response may be reversible, wherein removing a wearable UV sensor from a UV radiation source may reverse an induced photochromic response. For example, a UV sensor having a first color (including a colorless state) in the absence of UV radiation may reversibly change to a second color (including a colorless state) when exposed to UV radiation. For example, an initially colorless wearable UV sensor that has turned a color (e.g., purple) after an exposure to UV radiation may return to a colorless state upon removal of the wearable UV sensor from UV radiation exposure. A color change may be monitored (e.g., by instrumentation or human eye). For example, a color change may be monitored by a colorimeter or spectrophotometer. In some embodiments, an applied product may contact (e.g., by any desired manner of application including spreading, rubbing, smearing, coating, spraying, dipping or otherwise delivering such applied product) a wearable UV sensor. A wearable UV sensor may contact, receive, and/or otherwise interact with the applied product (e.g., in a manner similar, substantially similar, or the same as human skin). A wearable UV sensor may desirably be coated with, adsorb, or absorb an applied product in a substantially similar way as an animal (e.g., human) skin. A wearable UV sensor may desirably be comparable to human skin, wherein the wearable UV sensor may be coated by an applied product in the same way as the human skin. A wearable UV sensor may desirably be comparable to human skin, wherein the wearable UV sensor may be receive an applied product in the same way as the human skin. A wearable UV sensor may desirably be comparable to human skin, wherein the wearable UV sensor may retain an applied product in the same way as the human skin. A wearable UV sensor may desirably be comparable to human skin in features comprising tone, flexibility, and texture.

A substrate may comprise any materials desired or required for its intended or likely use. In some embodiments, a substrate may be an at least partially UV radiation-transmissible material, wherein a UV radiation exposure indicator may be molded into the substrate. A substrate may also be opaque. A substrate may also comprise a UV radiation exposure indicator coated onto at least one surface of the substrate. According to some embodiments, a polymer substrate may comprise at least one polymer. For example, a polymer substrate may comprise polyethylene, high density polyethylene, low density polyethylene, polypropylene, polyvinyl chloride, poly(methyl methacrylate), nylon, polytetrafluoroethene, polyurea, polystyrene, polysiloxane, polyacrylonitrile, copolymers thereof, or combinations thereof. For example, a wearable UV sensor may comprise a polymer substrate, wherein the polymer substrate is polyvinyl chloride. A combination of polymers may comprise a blend of more than one polymer or a series of more than one polymer layers. A polymer or combination of polymers may be chosen to provide a wearable UV sensor with desirable mechanical features comprising tensile strength, Young's Modulus, toughness, resilience, ductility, fracture strength, specific heat, heat retention, heat capacity, electrical conductivity, elongation, impact strength, tackiness, flexural modulus, softness, elongation at break, or combinations thereof. For example, a polymer substrate may desirably allow a wearable UV sensor to flexibly wrap around a wrist or to adhere as a thin strip to a surface (e.g., a skin). A polymer substrate may comprise a polymer or combination of polymers chosen to provide a wearable UV sensor with desirable color, opaqueness, permittivity of UV radiation, permittivity of electromagnetic radiation, or combinations thereof. A polymer substrate may selectively permit reflection, refraction, diffraction, transmission, adsorption, scattering, or combinations thereof, of specific wavelengths or broad spectrum electromagnetic radiation. For example, a polymer substrate may desirably allow a wearable UV sensor to test the exposure to UV-A radiation, UV-B radiation, or a combination thereof. In some embodiments, a polymer substrate may desirably allow a wearable UV sensor to simulate properties of human skin comprising texture, ability to absorb or retain an applied product, softness, hardness, or tone. A polymer substrate may desirably allow a wearable UV sensor to be breathable, flexible, tactile, and combinations thereof.

In some embodiments, a wearable UV sensor (e.g., a pericutaneous sensor, a dermal sensor) may comprise a polymer substrate, wherein the polymer substrate is present in any desired proportion, for example, at a concentration of about 5%, or of about 10%, or of about 15%, or of about 20% or of about 25%, or of about 30%, or of about 35%, or of about 40%, or of about 45%, or of about 50%, or of about 55%, or of about 60%, or of about 65%, or of about 70%, or of about 75%, or of about 80%, or of about 85%, or of about 95%, or of about 99%, in each case, by weight of the wearable UV sensor. A wearable UV sensor may comprise a polymer substrate present at a concentration from about 5% to about 10%, or from about 10% to about 20%, or from about 20% to about 30%, or from about 30% to about 40%, or from about 40% to about 50%, or from about 50% to about 60%, or from about 60% to about 70%, or from about 70% to about 80%, or from about 80% to about 90%, or from about 90% to about 99%, in each case, by weight of the wearable UV sensor. In some embodiments, a wearable UV sensor may comprise a polymer substrate present at a concentration from about 70% to about 90%, by weight of the wearable UV sensor.

A wearable UV sensor (e.g., a pericutaneous sensor, a dermal sensor), according to some embodiments, may comprise an adhesive. An adhesive may comprise a solvent based adhesive, a polymer dispersion adhesive, a hot-melt adhesive, a contact adhesive, a reactive adhesive, a structural adhesive, epoxy adhesives, hot melt adhesives, elastomeric adhesives, elastomeric contact adhesives, melamine formaldehyde adhesives, phenol formaldehyde adhesives, polyester resin adhesives, one-part adhesives, UV curing adhesives, moisture curing adhesives, heat curing adhesives, vegetable starch adhesives, casein based adhesives, cyanoacrylate adhesives, waterborne acrylic adhesives, spray adhesives, silicone based adhesives, or combinations thereof. An adhesive may desirably permit a wearable UV sensor to be applied to at least one surface. For example, an adhesive may desirably permit a wearable UV sensor to be applied to a skin of an animal (e.g., human). An adhesive may desirably permit a wearable UV sensor to be removably applied to a surface, wherein the wearable UV sensor may be applied multiple times. An adhesive may desirably permit a wearable UV sensor to be applied to a surface more than once without damaging the surface or the wearable UV sensor.

According to some embodiments, a wearable UV sensor (e.g., a pericutaneous sensor, a dermal sensor) may comprise a UV radiation exposure indicator. A wearable UV sensor may comprise at UV radiation exposure indicator, wherein the UV radiation exposure indicator is present in any desired proportion, for example, at a concentration of about 0.1%, or of about 1%, or of about 2%, or of about 5%, or of about 10%, or of about 15%, or of about 20% or of about 25%, or of about 30%, or of about 35%, or of about 40%, or of about 45%, or of about 50%, or more than about 50%, in each case, by weight of the wearable UV sensor. A wearable UV sensor may comprise a UV radiation exposure indicator present in any desired proportion, for example, at a concentration from about 1% to about 10%, or from about 10% to about 20%, or from about 20% to about 30%, or from about 30% to about 40%, or from about 40% to about 50%, or more than about 50%, in each case, by weight of the wearable UV sensor. A UV radiation exposure indicator may desirably indicate an exposure of a wearable UV sensor to UV radiation both quantitatively and qualitatively. In some embodiments, a wearable UV sensor may comprise a UV radiation exposure indicator present at a concentration from about 10% to about 30%, by weight of the wearable UV sensor.

In some embodiments, a wearable UV sensor (e.g., a pericutaneous sensor, a dermal sensor) may comprise an outer layer; a middle layer; and an inner layer. A wearable UV sensor may comprise an outer layer; a middle layer adjacent to the outer layer; and an inner layer adjacent to both the outer layer and the middle layer. An outer layer may comprise a substrate (e.g., a polymer substrate), a UV radiation exposure indicator, or combinations thereof. An outer layer may face away from a skin of a wearable UV sensor being worn on the skin. Facing away from a skin may desirably permit an outer layer to absorb a UV radiation. An outer layer may comprise a UV exposure indicator, wherein the outer layer may change color upon exposure of a threshold amount of a UV radiation. A middle layer may comprise a substrate (e.g., a polymer substrate) and an adhesive. A middle layer may or may not be exposed. For example, a middle layer may be covered by an outer layer or be covered by the outer layer and an inner layer. In some embodiments, a middle layer may be exposed in between an outer layer and the inner layer. A middle layer may desirably provide structural integrity to the outer layer. A middle layer may desirably adhere an outer layer to an inner layer. An inner layer may comprise an adhesive and a substrate (e.g., a polymer substrate). An inner layer may desirably adhere a top layer to a surface (e.g., a skin). In some embodiments, a wearable UV sensor may comprise a UV radiation exposure indicator as an outer layer; a polymer substrate as a middle layer; and an adhesive layer as an inner layer. A wearable UV sensor may comprise an outer layer, wherein the outer layer comprises a polymer substrate and UV radiation exposure indicator blend; and an inner layer comprising an adhesive. Having an adhesive strip as an inner layer and an outer layer comprising a UV radiation exposure indicator may desirably permit adhering or positioning a wearable UV sensor on a surface, wherein the UV radiation exposure indicator of the wearable UV sensor may be exposed to UV radiation. A wearable UV sensor may comprise an outer layer, wherein the outer layer comprises a substrate (e.g., a polymer substrate) and UV radiation exposure indicator blend; and an inner layer comprising a substrate (e.g., a polymer substrate).

In some embodiments, a wearable UV sensor (e.g., a pericutaneous sensor, a dermal sensor) may comprise an at least partially homogenized composition. An at least partially homogenized composition may comprise an adhesive. A wearable UV sensor may comprise an adhesive, wherein the adhesive is present in any desired proportion, for example, at a concentration of about 1%, or of about 5%, or of about 10%, or of about 15%, or of about 20% or of about 25%, or of about 30%, or of about 35%, or of about 40%, or of about 45%, or of about 50%, or of about 55%, or of about 60%, in each case, by weight of the wearable UV sensor. A wearable UV sensor may comprise an adhesive present in any desired proportion, for example, at a concentration from about 1% to about 10%, or from about 10% to about 20%, or from about 20% to about 30%, or from about 30% to about 40%, or from about 40% to about 50%, or from about 50% to about 60%, in each case, by weight of the wearable UV sensor. For example, a wearable UV sensor may comprise about 85% of a polymer, about 5% of an adhesive, and about 10% of a UV radiation exposure indicator.

A wearable UV sensor (e.g., a pericutaneous sensor, a dermal sensor) can have any regular or irregular shape or size. For example, a wearable UV sensor may be generally shaped as a circle, an oval, an asteroid, a monogon, a triangle, a square, a rectangle, a trapezium, a diamond, a rhombus, a parallelogram, a pentagon, a hexagon, a heptagon, an octagon, a nonagon, a decagon, or any other polygon. A wearable UV sensor may be substantially flat, or may have any 3-dimensional shape. In some embodiments, a wearable UV sensor may be sized to be worn on a human subject. For example, a wearable UV sensor may have the form of a ring, a bracelet, a wrist band, an arm band, a necklace, an earring, a belt, an article of clothing, a hat, sunglasses, or a combination thereof. A wearable UV sensor may be embedded within fabrics comprising neoprene, cotton, spandex, nylon, silk, wool, leather, hemp, jute, ramie, or combinations thereof. A wearable UV sensor may be a patch of any shape and size. For example, a wearable UV sensor may be a patch or test strip comprising a thickness from about 0.01 mm to about 30 mm. A wearable UV sensor may comprise a thickness of about 0.01 mm, or of about 0.05 mm, or of about 0.1 mm, or of about 0.25 mm, or of about 0.5 mm, or of about 0.75 mm, or of about 1 mm, or of about 2.5 mm, or of about 5 mm, or of about 7.5 mm, or of about 10 mm, or of about 20 mm, or of about 30 mm.

A wearable UV sensor may be a patch or test strip comprising a width from about 1 mm to about 700 mm. A wearable UV sensor may be a patch or test strip comprising a length from about 1 mm to about 700 mm. A wearable UV sensor may be a patch comprising a substantially circular shape and a thickness from about 0.01 mm to about 30 mm. A wearable UV sensor may be a patch comprising a substantially rectangular or polygonal shape, wherein the vertexes are substantially pointed or substantially rounded. According to some embodiments, a wearable UV sensor may comprise an area, the area comprising about 0.1 cm², or about 0.5 cm², or about 1 cm², or about 2 cm², or about 3 cm², or about 4 cm², or about 5 cm², or about 7.5 cm², or about 10 cm², or about 12.5 cm², or about 15 cm², or about 17.5 cm², or about 20 cm². For example, a wearable UV sensor may comprise an area of 1 cm².

A wearable UV sensor may comprise various textures, patterning, embossing, and prints. For example, a wearable UV sensor may be textured to be like a skin. A wearable UV sensor may comprise a texture, the texture comprising bumpy, firm, scratchy, slippery, stiff, fuzzy, rubbery, viscous, soft, grainy, squishy, fibrous, rough, silky, furry, hard, dense, coarse, spongy, sticky, flexible, squishy, or combinations thereof.

### UV Sensors

According to some embodiments, the present disclosure relates to UV sensors. A UV sensor may comprise a substrate (e.g., a polymer substrate); a UV radiation exposure indicator (e.g., photochromic dye); and optionally an adhesive. A UV sensor may be adapted for use adjacent to a person, wherein the person may desirably use the UV sensor to measure relative UV exposure of the person. For example, a UV sensor may be placed in a UV exposure area adjacent to a person. A UV sensor may have a shape of an umbrella, hat, canopy, or shelter, wherein the UV sensor may desirably measure relative UV exposure. Exposure of a UV sensor to UV radiation may induce a photochromic response in the UV sensor, wherein the magnitude of the photochromic response may correlate (e.g., directly correlate) to the magnitude or dose of the UV radiation exposure. A magnitude of a photochromic response may desirably permit measurement UV exposure relative to an adjacent person or object.

### UV Radiation Exposure Indicator

The present disclosure relates, according to some embodiments, to a UV sensor (e.g., a pericutaneous sensor, a dermal sensor) comprising a UV radiation exposure indicator. A UV radiation exposure indicator may desirably indicate an exposure of a UV sensor to UV radiation both quantitatively and qualitatively. A UV radiation exposure indicator may desirably differentiate between UV-A radiation and UV-B radiation. For example, a UV sensor comprising a UV radiation exposure indicator may qualitatively or quantitatively determine an exposure to UV-A radiation, UV-B, radiation, or a combination thereof. Differentiating between UV-A and UV-B radiation may be desirable because even though UV-A and UV-B radiation have been found to cause short term and long term damage to skin cells, UV-A radiation may be considered by health professionals to be more dangerous than UV-B to human skin. One reason for this may be because UV-A radiation may penetrate the skin surface, the epidermis and then into the dermis, whereas UV-B radiation does not typically penetrate the dermis, but may break through the skin surface and into the epidermis.

A UV radiation exposure indicator, according to some embodiments, may desirably indicate a UV radiation exposure of a UV sensor as a signal. A signal may comprise a chromatic change. A chromatic change (e.g., a color change) may be monitored (e.g., by instrumentation or human eye). For example, a chromatic change may be monitored by a colorimeter or spectrophotometer. A signal may be binary or ternary. For example, an UV radiation exposure indicator may change color upon exposure of a first threshold value of UV radiation and then change to a second color upon exposure of a second threshold value of UV radiation. A UV radiation exposure indicator may desirably indicate a UV radiation exposure of a UV sensor in a multitude of signals. For example, a UV radiation exposure indicator may indicate, upon exposure to a UV radiation, a UV index value from about 1 to about 11. In some embodiments, a chromatic change may comprise a change from colorless to a color, a change from a color to another color, a change from a first color to a second color and then back to the first color, a change from colorless to a color and then back to colorless, or combinations thereof. A chromatic change may comprise a hypsochromic shift, a bathochromic shift, a hyperchromic shift, a hypochromic shift, or a combination thereof. For example, a UV sensor comprising a UV exposure indicator may, upon exposure to threshold amount of a UV radiation, change from colorless to purple. A UV sensor that comprising a UV exposure indicator that has changed purple due to exposure to a threshold amount of a UV radiation may, upon removal of the UV exposure, change color form purple to colorless.

In some embodiments, a UV radiation exposure indicator comprises a photochromic dye (i.e., a photochromic pigment). A photochromic dye may be any chemical or combination of chemicals that exhibits a color change upon exposure to UV radiation. In some embodiments, upon exposure to UV radiation, a photochromic dye may exhibit a change in spectral band position in the absorption, reflectance, transmittance, or emission spectrum to a longer wavelength (e.g., bathochromic shift) or a shorter wavelength (e.g., hypsochromic shift). A photochromic dye may comprise spiro-oxazines, spiro-indoline-oazines, spiropyrans, diarylethylenes, azobenzenes, photochromic quinones, inorganic photochromics, photochromic coordination compounds, or combinations thereof. For example, a photochromic dye may comprise 1′,3′-dihydro-8-methoxy-1′,3′,3′-trimethyl-6-nitrospiro[2H-1-benzopyran-2,2′-(2H)-indole], benzyl viologen dichloride, 1,1′-diheptyl-4,4′-bipyridinium dibromide, 1′,3′-dihydro-1′,3′,3′-trimethyl-6-nitrospiro[2H-1-benzopyran-2,2′-(2H)-indole], 1,3-dihydro-1,3,3-trimethylspiro[2H-indole-2,3′-[3H] naphtha[2,1-b][1,4] oxazine], 4,4′-dipyridyl, indoine blue dye content, methyl viologen dichloride hydrate, 2,3,3-trimethyl-1-propyl-3H-indolium iodide, azobenzene, silver halide, zinc halide, yttrium hydride, stilbene, 1,2-di(thiophen-3-yl)cyclopent-1-ene, 2,2-diphenyl-2H-benzo[h]chromene, dimethyl pyrene, 2-(4-(8-(4-(4,5-diphenyl-2H-imidazol-2-yl)phenyl)naphthalen-1-yl)phenyl)-4,5-diphenyl-1H-imidazole, or combinations thereof. A photochromic dye may comprise 1,3-dihydrospiro[2H-anthra[2,3-d]imidazole-2, cyclohexane]-5,10-dione; 2-Nitro-3-aminofiuoren-2-amino-4-(2′-furanyl)-6H-1,3-thiazine-6-thione, 1,3-dihydro-4-(phenyl thio) spiro[2H-anthra ‘1,2-diimidazole-2,1’-cyclohexane]-6,11-dione, 1,3-dihydrospiro[2-H-anthra[1,2-dimidazole-2,1′cycloheptane]-6,11-diane, 8-methoxy-1′,3′-dimethylspiro(2H-1-benzopyran-2,2′(1′H)-quinoline, (1,3-propanediyl)bis[3′,3′-dimethyl-6-nitrospiro[2H-1-benzopyran-2,2′-indoline], or combinations thereof. A photochromic dye may comprise 1,3,3-trimethylspiro-indole-2,3′-[3H]-naphtha[2,1-b]-1,4-oxazine], spiro[2H-1-benzopyran-2,9′-xanthene], 5′-amino-l′,3′,3′-trimethylspiro[2H-1-benzopyran-2,2′indoline, 3,3′-dimethyl-6-nitrospiro[2H-1-benzopyrao-2,2′benzoxazoline], [(2′,4′-dinitrophenyl)methy 1]-1H-benzimidazole, 2,5-diphenyl-4-(2′-chlorophenyl)imidazole, 6′-methylthio-3,3′-dimethyl-8-methoxy-6-nitrospiro[2H-1-benzopyran-2,2′-benzothiozoline], (1,2-ethanediyl)bis[8-methoxy-3-methyl-6-nitrospiro[2H-1-benzopyran-2,2′-benzothiozoline]], 2-phenyl-3-methy 1-7-methoxy-8′-nitrospiro[4H-benzopyran-4,3′-[3H]-naphtho]2,1-b]pyran, 1,3-dihydrospiro[2H-anthra[2,3-d]imidazole-2,1′cyclohexane]-6,11-dione, ethyl-(3-methyl-(3-(3′,3′-dimethy-1-6-nitrospiro (2H-1-benzopyran-2,2′-indolin-1′-yl)-propenoate-3,4,4-tetrachloronaphthalen-1(4H)-one, (E)-3-(1-(furan-3-yl) ethylidene)-4-(propan-2-ylidene) dihydrofuran-2,5-dione, or combinations thereof. According to some embodiments, a photochromic dye or combinations of photochromic dyes may desirably change to a color having a strong contrast to a typical skin. For example, a dye may desirably change to a color comprising red, orange, yellow, green cyan, blue, purple, or violet. Different combinations of dyes may desirably provide for UV exposure sensors to change from colorless or white to various colors.

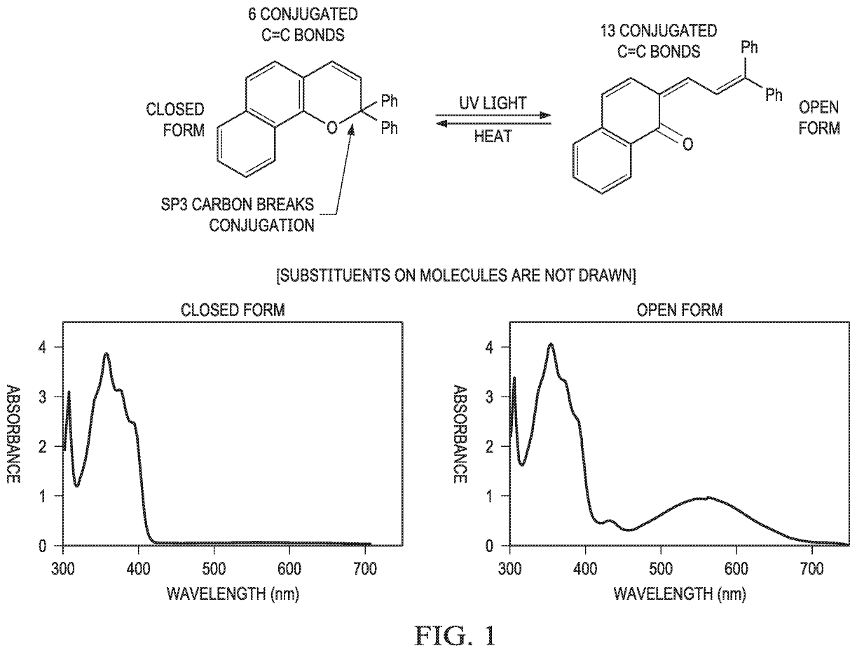

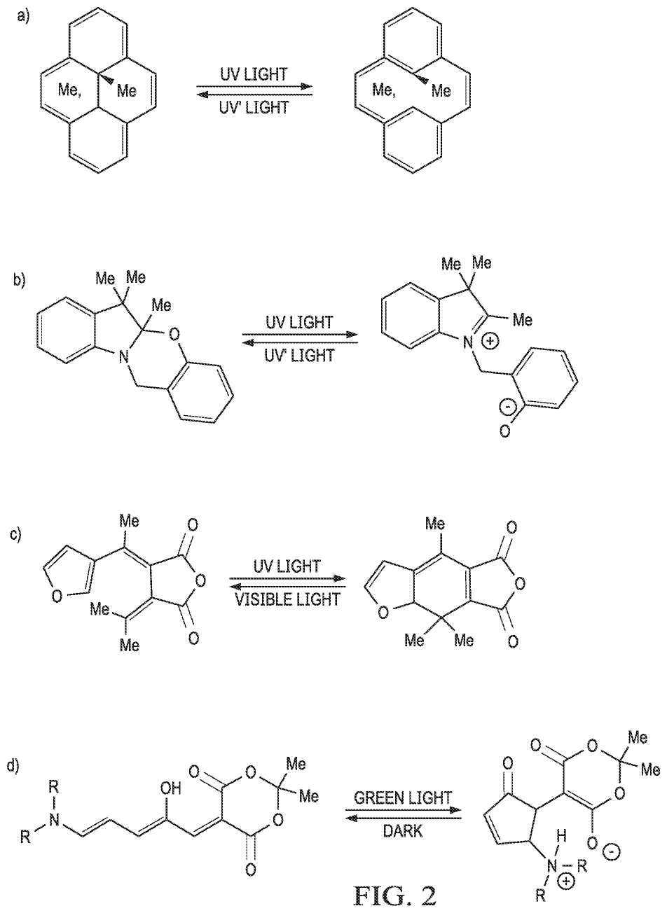

According to some embodiments, a photochromic dye or pigment can produce a color change upon exposure to UV radiation through intramolecular or intermolecular rearrangements, reactions, or combinations thereof. As shown in FIG. 1, 2,2-diphenyl-2H-benzo[h]chromene (“closed form”) may undergo an intramolecular rearrangement to form (Z)-2-(3,3-diphenylallylidene)naphthalen-1(2H)-one (“open form”) upon exposure to UV radiation, wherein the rearrangement may be reversed through exposure to heat. The closed form structure is colorless, whereas, upon UV radiation exposure, the structure rearranges to the open form, which is purple. This is evident from the absorbance spectrum for each form. Additional rearrangements may occur in different molecules through exposure of different wavelengths of electromagnetic radiation and/or heat. For example, FIG. 2 discloses intramolecular rearrangements including a) trans-10b,10c-Dimethyl-10b,10c-dihydropyrene, b) 5a,6,6-trimethyl-5a,6-dihydro-12H-benzo[5,6][1,3]oxazino[3,2-a]indole, c) (E)-3-(1-(furan-3-yl) ethylidene)-4-(propan-2-ylidene)dihydrofuran-2,5-dione, and d) 5-((2Z,4E)-5-(dimethylamino)-2-hydroxypenta-2,4-dien-1-ylidene)-2,2-dimethyl-1,3-dioxane-4,6-dione.

### UV Radiation Exposure

According to some embodiments, the present disclosure relates to dermal sensor comprising a polymer substrate; a UV radiation exposure indicator (i.e., photochromic dye); and an adhesive, wherein exposing the wearable UV sensor to a dose UV radiation may induce a photochromic response in the wearable UV sensor. A dose may vary upon the intensity and length of exposure of a wearable UV sensor to UV radiation. For example, a wearable UV sensor may receive a larger dose of UV radiation if exposed for a longer time than a corresponding wearable UV sensor exposed for a shorter time. A dose may be reduced through an application of an applied product (e.g., sun blocks, UVA filters, or UVB filters). For example, a wearable UV sensor coated with an applied product may receive a lower dose of UV radiation than a corresponding wearable UV sensor not coated with an applied product or coated with a different applied product with a lower solar protection factor (SPF). A photochromic response may vary according to dose. For example, a wearable UV sensor may undergo a photochromic response by changing to a shade of purple upon receiving a dose of a UV radiation, wherein the wearable UV sensor may turn a darker or deeper shade of purpose upon receiving a larger dose of the UV radiation. A wearable UV sensor may be formulated to exhibit a photochromic response with selectivity with respect to UV radiation wavelength ranges. For example, a wearable UV sensor may exhibit a photochromic response upon receiving a dose of UV-A radiation, wherein the wearable UV sensor may not exhibit a photochromic response upon receiving a dose of UV-B radiation. A wearable UV sensor may exhibit a photochromic response in gradients or in a binary fashion upon being treated with a dose of UV radiation. For example, a wearable UV sensor may, upon exposure to a dose of UV radiation, begin to turn a light shade of purple, wherein the wearable UV sensor will turn a darker purple in a gradient directly correlated to the dose of UV-radiation. In some embodiments, a wearable UV sensor may have a photochromic response upon reaching a threshold value of UV radiation. For example, a wearable UV sensor may remain colorless until reaching a threshold value of UV radiation exposure, then upon reaching the threshold value of UV radiation exposure, change colors. A threshold value may comprise a sum of UV radiation exposure, a sum of a length of UV radiation exposure, a sum of an intensity of UV radiation exposure, or a combination thereof. An exposure may comprise direct or indirect exposure of a wearable UV sensor to UV radiation.

According to some embodiments, a UV radiation exposure indicator may indicate a gradient or threshold UV radiation exposure level. For example, a UV radiation exposure indicator may indicate a threshold UV radiation energy density in Joules per cm², or a threshold irradiance Watts per cm². A UV radiation exposure indicator can be tuned or adjusted to sense or indicate varying degrees of exposure. For example, mixture of photochromic dyes can be combined in varying formulations to express photochromic responses at different threshold UV radiation exposures. In some embodiments, a UV radiation exposure indicator may desirably sense the UV radiation exposure in real time. For example, a UV radiation exposure indicator may desirably sense the efficacy of an applied product (e.g., sun blocks, UVA filters, or UVB filters) to protect against the UV radiation in real time.

### Methods for Determining UV Radiation Protection Efficacy of an Applied Product

The present disclosure relates, according to some embodiments, to methods for determining a UV radiation protection efficacy. A wearable UV sensor (e.g., a pericutaneous sensor, a dermal sensor) may be coated with an applied product (e.g., sun blocks, UVA filters, or UVB filters) and then exposed to UV radiation, wherein the wearable UV sensor may provide a photochromic response upon reaching a threshold exposure value of UV radiation. A wearable UV sensor may exhibit a photochromic response which may quantitatively or qualitatively measure an ability for an applied product or sunscreen to absorb or reflect UV radiation. A photochromic response may indicate a breakdown in an ability or a reduction in the efficacy of the ability for an applied product to absorb or reflect UV radiation. For example a wearable UV sensor coated with an applied product may not initially exhibit a photochromic response upon exposure to a UV radiation, but may exhibit the photochromic response upon an extended exposure to the UV radiation, which may be due to a breakdown of the ability for the applied product to absorb or reflect UV radiation. An efficacy for an applied product to absorb or reflect UV radiation may be measured by a response of a delayed or a persistent photochromic response of a wearable UV sensor coated with the applied product.

A method for sensing a threshold dose of a UV radiation exposure, the method comprising providing a wearable UV sensor comprising a polymer substrate and a UV radiation exposure indicator; exposing the wearable UV sensor to at least one dose of the UV radiation to form an irradiated wearable UV sensor; and sensing the threshold dose of the UV radiation with a photochromic response by the irradiated wearable UV sensor, wherein the photochromic response results from exposure of UV radiation comprising a wavelength from about 290 nm to about 400 nm.

A method for determining a magnitude of a UV radiation protection of an applied product, the method comprising: (a) providing a dermal sensor comprising a polymer substrate, a UV radiation exposure indicator, and an adhesive layer; (b) applying at least one coat of the UV protector to a surface of the dermal sensor to produce a UV protector coated dermal sensor; (c) exposing the UV protector coated dermal sensor to at least one dose of a UV radiation to form an irradiated dermal sensor, wherein the exposing results in a photochromic response by the dermal sensor; and (d) measuring the photochromic change relative to a reference to determine the magnitude of the UV radiation protection of the applied product by, wherein the first dose of UV radiation comprises a wavelength range from about 290 nm to about 400 nm. Measuring a level of photochromic response may comprise measuring by instrumentation or human eye. For example, instrumentation may comprise a colorimeter or spectrophotometer.

A method may comprise sensing a threshold dose of an electromagnetic radiation. A dose of electromagnetic radiation may comprise a wavelength of greater than or equal to 10 m, or from about 10 cm to about 10 m, or from about 1 mm to about 10 cm, or from about 30 μm to about 1 mm, or from about 3 μm to about 30 μm, or from about 700 nm to about 3 μm, or form about 400 nm to about 700 nm, or from about 200 nm to about 400 nm, or from about 280 nm to about 290 nm, from about 290 nm to about 400 nm, or about 10 nm to about 200 nm, or about 0.01 nm to about 10 nm, or combinations thereof. In some embodiments, a dose of electromagnetic radiation may comprise exposure with a fixed wavelength or wavelength range or a varying wavelength or wavelength range of electromagnetic radiation over a time, the time comprising from about 1.2×10⁻⁷ s to about 1 s, or from about 1 s to about 60 s, or from about 60 s to about 1 hours, or form about 1 hour to about 4 hours, or from about 4 hours to about 8 hours, or from about 8 hours to about 12 hours, or from about 12 hours to about 16 hours, or from about 16 hours to about 24 hours. A duration of time exposure may be non-stop, intermittent, fixed interval, or combinations thereof.

A method for determining a UV radiation protection efficacy may comprise comparing a first wearable UV sensor coated with an applied product to a second strip not coated an applied product. In some embodiments, a method for determining a UV radiation protection efficacy may comprise comparing a first wearable UV sensor coated with an applied product to a second strip not coated an applied product. A method of determining a UV radiation protection efficacy may comprise comparing a wearable UV sensor that has been exposed to UV radiation or not exposed to UV radiation to a scale, ladder, comparison chart, or a combination thereof. A scale, ladder, or comparison chart may depict a result of a wearable UV sensor at different UV radiation exposure levels. For example, a scale, ladder, or comparison chart may depict a series of wearable UV sensors comprising a gradient of shades of purple from light purple to dark purple, which directly correlate to a dose response of a low dose UV radiation to a high dose of UV radiation. A scale, ladder, or comparison chart may comprise multiple colors that directly correlate to UV radiation exposure levels. For example, a colorless wearable UV sensor may correlate to no UV exposure, a green wearable UV sensor may correlate to the low dose UV exposure, and a red wearable UV sensor may correlate to a high dose UV exposure.

### Systems for Sensing UV Radiation Exposure

According to some embodiments, the present disclosure relates to a system for sensing UV radiation exposure may comprise a plurality of wearable UV sensors, a UV radiation source, and a chart, wherein the chart may be used to compare UV radiation exposure dose levels of the wearable UV sensors. A system may comprise a single wearable UV sensor and a scale. A system may comprise at least one wearable UV sensor and a scale, wherein the wearable UV sensor may be single use or disposable.

A system for sensing at least one threshold dose of a UV radiation may comprise a wearable UV sensor, wherein the wearable UV sensor comprises a polymer substrate. A polymer substrate may comprise polyethylene, high density polyethylene, low density polyethylene, polypropylene, polyvinyl chloride, poly(methyl methacrylate), nylon, polytetrafluoroethene, polyurea, polystyrene, polysiloxane, polyacrylonitrile, copolymers thereof, or combinations thereof. A polymer substrate may be in the form of a resin. For example, a resin may comprise polyvinyl chloride and a solvent comprising methyl ethyl ketone, tetrahydrofuran, acetone, cyclohexanone, or combinations thereof. A polymer substrate may be present at a concentration from about 5% to about 10%, or from about 10% to about 20%, or from about 20% to about 30%, or from about 30% to about 40%, or from about 40% to about 50%, or more than about 50%, in each case, by weight of the at least one wearable UV sensor.

A dermal sensor system for sensing at least one threshold dose of a UV radiation may comprise a dermal sensor, wherein the dermal sensor comprises an adhesive comprising a solvent based adhesive, a polymer dispersion adhesive, a hot-melt adhesive, a contact adhesive, a reactive adhesive, a structural adhesive, an epoxy adhesive, a hot melt adhesive, an elastomeric adhesive, an elastomeric contact adhesive, a melamine formaldehyde adhesive, a phenol formaldehyde adhesive, a polyester resin adhesive, a one-part adhesive, a UV curing adhesive, a moisture curing adhesive, a heat curing adhesive, a vegetable starch adhesive, a casein based adhesive, a cyanoacrylate adhesive, a spray adhesive, a silicone based adhesive, or a combination thereof. An adhesive may be present at a concentration from about 1% by weight to about 10% by weight, or from about 10% by weight to about 20% by weight, or from about 20% by weight to about 30% by weight, or from about 30% by weight to about 40% by weight, or from about 40% by weight to about 50% by weight, or from about 50% by weight to about 60% by weight, or from about 60% by weight to about 70% by weight, or from about 70% by weight to about 80% by weight, or from about 80% by weight to about 90% by weight, or from about 90% by weight to about 99% by weight, by weight of the dermal sensor.

A system for sensing at least one threshold dose of a UV radiation may comprise a wearable UV sensor comprising a UV radiation exposure indicator comprising a photochromic dye comprising spiro-oxazines, spiro-indoline-oazines, spiropyrans, diarylethylenes, azobenzenes, photochromic quinones, inorganic photochromics, photochromic coordination compounds, or combinations thereof. A UV radiation exposure indicator may be present at a concentration from about 1% by weight to about 10% by weight, or from about 10% by weight to about 20% by weight, or from about 20% by weight to about 30% by weight, or from about 30% by weight to about 40% by weight, or from about 40% by weight to about 50% by weight, or from about 50% by weight to about 60% by weight, or from about 60% by weight to about 70% by weight, or from about 70% by weight to about 80% by weight, or from about 80% by weight to about 90% by weight, or from about 90% by weight to about 99% by weight, by weight of the at least one wearable UV sensor.

A chart may comprise a color gradient, wherein the color gradient corresponds to a UV exposure dose photochromic response. For example, a chart may comprise a color gradient that transitions from a first color to a second color, wherein each step of the color gradient is labeled with a relative UV exposure level. In some embodiments, a UV radiation exposure indicator having a color similar to a first color of a color gradient may have received little to no UV radiation exposure, wherein a corresponding UV radiation exposure indicator having a color similar to a second color of the color gradient may have received a high UV radiation exposure relative to the UV radiation exposure indicator having the color similar to the first color of the color gradient.

A system for sensing at least one threshold dose of a UV radiation may comprise a pericutaneous sensor in the shape of a ring, a bracelet, a wrist band, an arm band, a necklace, an earring, a belt, an article of clothing, a hat, sunglasses, or a combination thereof.

### SPECIFIC EXAMPLE EMBODIMENTS

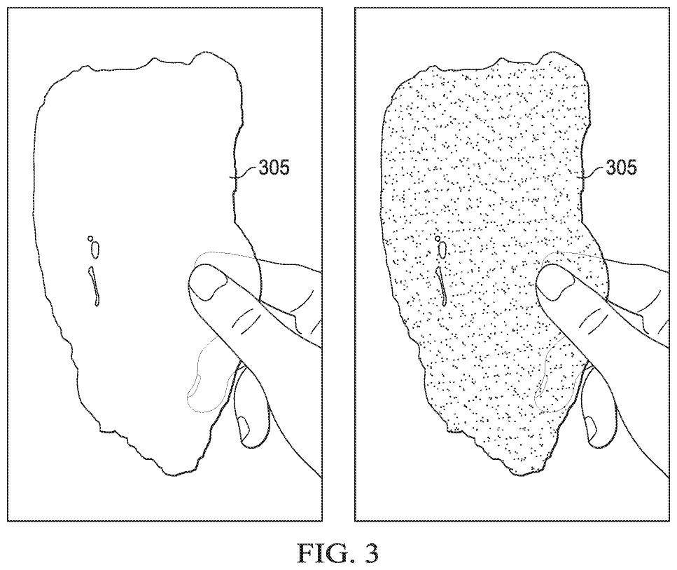

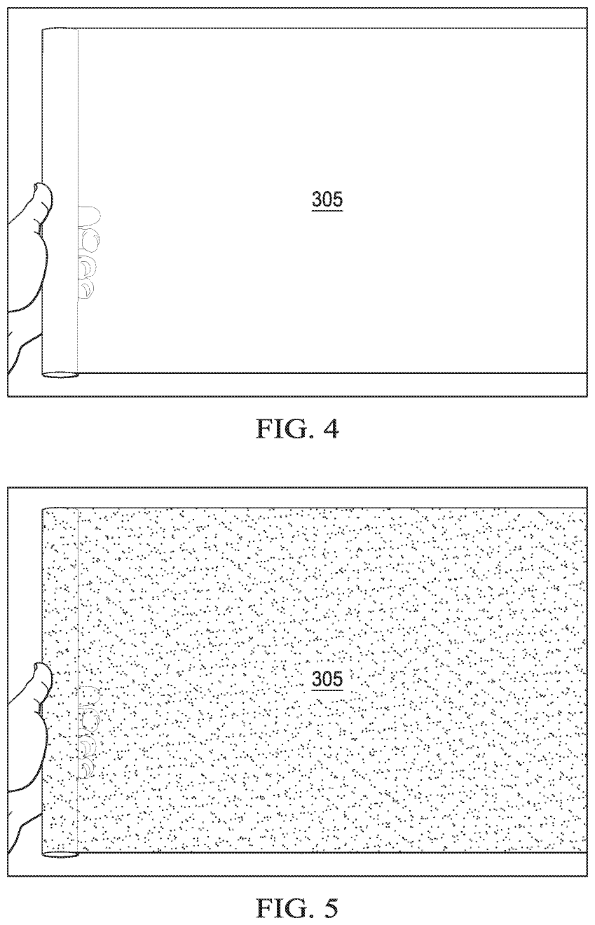

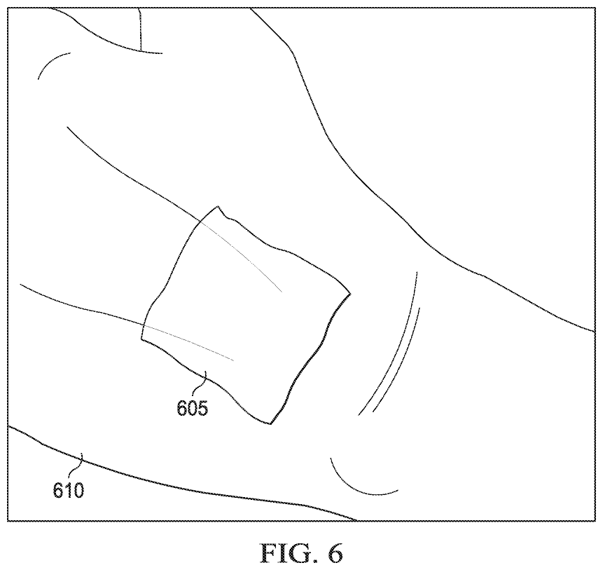

FIGS. 3-5 illustrate a wearable UV sensor in the presence and in the absence of ultraviolet radiation according to a specific example embodiment of the disclosure. A wearable UV sensor may comprise a polymer comprising polyvinyl chloride and a UV radiation exposure indicator. A wearable UV sensor may be white or colorless in the absence of UV radiation. When exposed to light, a wearable UV sensor may turn purple, wherein the wearable UV sensor may turn white again when removed from the UV radiation source. As shown in FIG. 6, at least a portion of a wearable UV sensor may desirably be adhered to a human skin, wherein the wearable UV sensor is substantially flat with respect to the human skin.

As will be understood by those skilled in the art who have the benefit of the instant disclosure, other equivalent or alternative compositions and methods for qualitatively or quantitatively measuring radiation exposure can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Where open terms such as “having” or “comprising” are used, one of ordinary skill in the art having the benefit of the instant disclosure will appreciate that the disclosed features or steps optionally may be combined with additional features or steps. Such option may not be exercised and, indeed, in some embodiments, disclosed systems, compositions, apparatuses, and/or methods may exclude any other features or steps beyond those disclosed herein. Elements, compositions, devices, systems, methods, and method steps not recited may be included or excluded as desired or required. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value +/− about 10%, depicted value +/− about 50%, depicted value +/− about 100%) and/or a range endpoint. With respect to the former, a value of 50 depicted in an example, table, and/or drawing may form the basis of a range of, for example, about 45 to about 55, about 25 to about 100, and/or about 0 to about 100. Disclosed percentages are weight percentages except where indicated otherwise.

All or a portion of a device and/or system for compositions and methods for qualitatively or quantitatively measuring radiation exposure may be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

### EXAMPLES

Some specific example embodiments of the disclosure may be illustrated the examples provided herein.

### Example 1: Formulating Wearable UV Sensor

A general process of formulating a wearable UV sensor is described in this example. A PVC solution comprising a resin of highly polymerized PVC and cyclohexanone was combined with a spiro-mero photochromism material. The ratio of PVC to dye was about 70:30. Then, the PVC and dye mixture was applied onto a polyethylene terephthalate (PET) substrate. Substantially all cyclohexanone was then evaporated to produce a solidified PVC film. A waterborne acrylic adhesive was then applied onto the solidified PVC film. An attach release paper was applied on top of the adhesive. Next, the PET substrate was peeled away from the PVC film and the PVC film was then wrapped up into a roll.

What is claimed is:

1. A wearable UV sensor for detecting a threshold dose of a UV radiation, the wearable UV sensor comprising:
(a) a first layer comprising a polymer substrate comprising a UV radiation exposure indicator; and
(b) a second layer comprising an adhesive;
wherein exposure of the UV radiation exposure indicator to the threshold dose of the UV radiation produces a photochromic response in the UV radiation exposure indicator, wherein the wearable UV sensor is sized to be worn on a human subject,
wherein the UV radiation comprises a wavelength range from about 290 nm to about 400 nm, and wherein the wearable UV sensor is configured to receive an applied product in a manner comparable to human skin.

2. A wearable UV sensor for detecting a dose of a UV radiation, the wearable UV sensor comprising:
(a) a first layer comprising a polymer substrate; and
(b) a second layer comprising a UV radiation exposure indicator;
wherein exposure of the UV radiation exposure indicator to the dose of the UV radiation produces a photochromic response in the UV radiation exposure indicator,
wherein the wearable UV sensor is sized to be worn on a human subject, and
wherein the UV radiation comprises a wavelength range from about 290 nm to about 400 nm.

3. The wearable UV sensor according to claim 2, further comprising a third layer, the third layer comprising an amount of an adhesive sufficient to adhere at least a portion of the wearable UV sensor to human skin.

4. The wearable UV sensor according to claim 2, wherein the polymer substrate comprises polyethylene, high density polyethylene, low density polyethylene, polypropylene, polyvinyl chloride, poly(methyl methacrylate), nylon, polytetrafluoroethene, polyurea, polystyrene, polysiloxane, polyacrylonitrile, copolymers thereof, or combinations thereof.

5. The wearable UV sensor according to claim 2, wherein the UV radiation exposure indicator comprises a photochromic dye comprising spiro-oxazines, spiro-indoline-oazines, spiropyrans, diarylethylenes, azobenzenes, photochromic quinones, inorganic photochromics, photochromic coordination compounds, or combinations thereof.

6. The wearable UV sensor according to claim 3, wherein the adhesive comprises a solvent based adhesive, a polymer dispersion adhesive, a hot-melt adhesive, a contact adhesive, a reactive adhesive, a structural adhesive, an epoxy adhesive, a hot melt adhesive, an elastomeric adhesive, an elastomeric contact adhesive, a melamine formaldehyde adhesive, a phenol formaldehyde adhesive, a polyester resin adhesive, a one-part adhesive, a UV curing adhesive, a moisture curing adhesive, a heat curing adhesive, a vegetable starch adhesive, a casein based adhesive, a cyanoacrylate adhesive, a spray adhesive, a silicone based adhesive, or a combination thereof.

7. The wearable UV sensor of claim 2, wherein the polymer substrate is present at a concentration from about 70% by weight to about 90% by weight of the wearable UV sensor.

8. The wearable UV sensor of claim 2, wherein the UV radiation exposure indicator is present at a concentration from about 10% by weight to about 30% by weight of the wearable UV sensor.

9. The wearable UV sensor of claim 3, wherein the adhesive is present at a concentration from about 1% by weight to about 10% by weight of the wearable UV sensor.

10. The wearable UV sensor of claim 2, wherein the wearable UV sensor has a shape of a ring, a bracelet, a wrist band, an arm band, a necklace, an earring, a belt, an article of clothing, a hat, sunglasses, or a combination thereof.

11. The wearable UV sensor of claim 2, wherein the wearable UV sensor further comprises an area from about 0.1 cm² to about 5 cm².

12. The wearable UV sensor of claim 2, wherein the dose of a UV radiation is a threshold dose.

13. A dermal sensor for detecting a dose of a UV radiation, the dermal sensor comprising:
(a) a polymer substrate comprising at least one polymer and at least one UV radiation exposure indicator; and
(b) an adhesive,
wherein exposure of the dermal sensor to the dose of the UV radiation produces a photochromic response in the polymer substrate,
wherein the dermal sensor is sized to be worn on a human subject, and
wherein the UV radiation comprises a wavelength range from about 290 nm to about 400 nm.

14. The dermal sensor according to claim 13, wherein the at least one polymer is selected from the group consisting of polyethylene, high density polyethylene, low density polyethylene, polypropylene, polyvinyl chloride, poly(methyl methacrylate), nylon, polytetrafluoroethene, polyurea, polystyrene, polysiloxane, polyacrylonitrile, and copolymers thereof.

15. The dermal sensor according to claim 13, wherein the at least one UV radiation exposure indicator comprises a photochromic dye comprising spiro-oxazines, spiro-indoline-oazines, spiropyrans, diarylethylenes, azobenzenes, photochromic quinones, inorganic photochromics, photochromic coordination compounds, or combinations thereof.

16. The dermal sensor according to claim 13, wherein the adhesive comprises a solvent based adhesive, a polymer dispersion adhesive, a hot-melt adhesive, a contact adhesive, a reactive adhesive, a structural adhesive, an epoxy adhesive, a hot melt adhesive, an elastomeric adhesive, an elastomeric contact adhesive, a melamine formaldehyde adhesive, a phenol formaldehyde adhesive, a polyester resin adhesive, a one-part adhesive, a UV curing adhesive, a moisture curing adhesive, a heat curing adhesive, a vegetable starch adhesive, a casein based adhesive, a cyanoacrylate adhesive, a spray adhesive, a silicone based adhesive, or a combination thereof.

17. The dermal sensor of claim 13, wherein the at least one polymer is present at a concentration from about 70% by weight to about 90% by weight of the polymer substrate.

18. The dermal sensor of claim 13, wherein the UV radiation exposure indicator is present at a concentration from about 10% by weight to about 30% by weight of the polymer substrate.

19. The dermal sensor of claim 13, wherein the adhesive is present at a concentration from about 1% by weight to about 10% by weight of the dermal sensor.

20. The dermal sensor of claim 13, wherein the dermal sensor further comprises an area from about 0.1 cm² to about 5 cm².

21. The dermal sensor of claim 13, wherein the dose of a UV radiation is a threshold dose.

22. A system for sensing at least one dose of a UV radiation, the system comprising:
(a) at least one dermal sensor comprising a polymer substrate, at least one UV radiation exposure indicator, and an adhesive; and
(b) a chart comprising a UV radiation dose response,
wherein the at least one dermal sensor is configured to undergo a photochromic response when sensing the at least one dose of the UV radiation,
wherein the UV radiation comprises a wavelength from about 290 nm to about 400 nm, and wherein the at least one dermal sensor is sized to be worn on a human subject.

23. The system according to claim 22, wherein the polymer substrate comprises polyethylene, high density polyethylene, low density polyethylene, polypropylene, polyvinyl chloride, poly(methyl methacrylate), nylon, polytetrafluoroethene, polyurea, polystyrene, polysiloxane, polyacrylonitrile, copolymers thereof, or combinations thereof.

24. The system according to claim 22, wherein the at least one UV radiation exposure indicator comprises a photochromic dye comprising spiro-oxazines, spiro-indoline-oazines, spiropyrans, diarylethylenes, azobenzenes, photochromic quinones, inorganic photochromics, photochromic coordination compounds, or combinations thereof.

25. The system according to claim 22, wherein the adhesive comprises a solvent based adhesive, a polymer dispersion adhesive, a hot-melt adhesive, a contact adhesive, a reactive adhesive, a structural adhesive, an epoxy adhesive, a hot melt adhesive, an elastomeric adhesive, an elastomeric contact adhesive, a melamine formaldehyde adhesive, a phenol formaldehyde adhesive, a polyester resin adhesive, a one-part adhesive, a UV curing adhesive, a moisture curing adhesive, a heat curing adhesive, a vegetable starch adhesive, a casein based adhesive, a cyanoacrylate adhesive, a spray adhesive, a silicone based adhesive, or a combination thereof.

26. The system according to claim 22, wherein the at least one polymer substrate is present at a concentration from about 50% by weight to about 90% by weight of the at least one wearable UV sensor.

27. The system of claim 22, wherein the UV radiation exposure indicator is present at a concentration from about 1% by weight to about 10% by weight of the at least one wearable UV sensor.

28. The system of claim 22, wherein the adhesive is present at a concentration from about 1% by weight to about 10% by weight of the at least one wearable UV sensor.

29. The system of claim 22, wherein the at least one dermal sensor may comprise a pericutaneous sensor comprising the shape of a ring, a bracelet, a wrist band, an arm band, a necklace, an earring, a belt, an article of clothing, a hat, sunglasses, or a combination thereof.

30. The system of claim 22, wherein the at least one dermal sensor further comprises an area from about 0.1 cm² to about 5 cm².

31. The system of claim 22, wherein the UV radiation dose response comprises a color gradient, wherein the color gradient corresponds to a UV exposure dose photochromic response.
